(19) United States
(12) Patent Application Publication
(10) Pub. No.: US 2021/0121624 A1
(43) Pub. Date: Apr. 29, 2021

(54) IMPLANTABLE INFUSION DEVICES WITH CLOSED LOOP SENSING AND ASSOCIATED METHODS

(71) Applicant: The Alfred E. Mann Foundation for Scientific Research, Valencia, CA (US)

(72) Inventors: Robert J. GREENBERG, Los Angeles, CA (US); Harshit R. Suri, Pasadena, CA (US); Thomas J. Lobl, Santa Clarita, CA (US); Brian M. Shelton, Altadena, CA (US); Sam W. Bowman, Santa Clara, CA (US)

(73) Assignee: The Alfred E. Mann Foundation for Scientific Research, Valencia, CA (US)

(21) Appl. No.: 17/078,816

(22) Filed: Oct. 23, 2020

Related U.S. Application Data
(60) Provisional application No. 62/925,223, filed on Oct. 23, 2019.

(51) Int. Cl. A61M 5/142

(52) U.S. Cl. CPC A61M 5/14276; A61M 2205/18; A61M 2205/3553; A61M 2230/50; A61M 2230/20; A61M 2230/04; A61M 2230/42

(57) ABSTRACT

Implantable infusion devices and methods that provide closed loop control.

### CROSS-REFERENCE TO RELATED APPLICATIONS

This application claims the benefit of and priority to previously filed U.S. Provisional Patent Application Ser. No. 62/925,223, filed Oct. 23, 2019, which is entitled “Implantable Pump With Closed Loop Sensing” and incorporated herein by reference in its entirety.

### BACKGROUND OF THE INVENTIONS

### 1. Field of Inventions

The present inventions relate generally to implantable infusion devices.

### 2. Description of the Related Art

Implantable infusion devices are used to provide patients with a medication or other substance (collectively “infusible substance”) and frequently include a reservoir and a pump or other fluid transfer device. The reservoir is used to store the infusible substance and, in some instances, implantable infusion devices are provided with a refill port that allows the reservoir to be transcutaneously filled (and/or re-filled) with a hypodermic needle. The reservoir is coupled to the fluid transfer device, which is in turn connected to an outlet port. A catheter, which has an outlet at the target body region, may be connected to the outlet port. As such, infusible substance from the reservoir may be transferred from the reservoir to the target body region by way of the fluid transfer device and catheter.

Implantable infusion devices are used in a wide variety of treatments. One exemplary application is the treatment of pain. Chronic pain, which may result from spinal disorders such as post-lam inectomy syndrome, compression fractures, spinal stenosis, spondylosis and spondylolisthesis, as well as non-spine-related pain disorders such as complex regional pain syndrome, rheumatoid arthritis, connective tissue disorders and chronic pancreatitis, is one type of pain that may be treated with an implantable infusion device. Cancer-related pain is another type of pain that may be treated with an implantable infusion device. In at least some instances, the infusion device is implanted in a subcutaneous pocket in the lower abdominal region. Opioid or non-opioid pain medication (collectively “pain medication”) is delivered to the subarachnoid space in accordance with a stored delivery profile by way of a catheter that extends from the infusion device. The stored delivery profile may, for example, be configured to provide pain medication dosages that increase and decrease over a 24-hour period based on expected patient activity.

Although implantable infusion devices have proven effective in that they allow patients to forgo oral opioids and their harmful side effects, the present inventors have determined that implantable infusion devices are susceptible to improvement. For example, conventional implantable infusion devices operating in accordance with a stored delivery profile cannot in real time adjust the dosage to be delivered based on the actual state of the patient. In the exemplary context of pain treatment, conventional implantable infusion devices operating in accordance with a stored delivery profile cannot automatically increase delivery of pain medication in response to unexpected increases in pain. Conventional implantable infusion device operating in accordance with a stored delivery profile also cannot automatically cease delivery of pain medication in response to overdose including, but not limited to, an overdose caused by the patient's use of opioids beyond those provided by the infusion device (e.g., oral opioids).

### SUMMARY OF THE INVENTIONS

A method in accordance with one embodiment of a present invention includes the steps of delivering pain medication to a patient with an implantable infusion device, monitoring biometric data of the patient with a patient monitor, determining, with the implantable infusion device, whether or not the monitored biometric data is indicative of an opioid overdose, and immediately ending delivery of the pain medication with the implantable infusion device in response to a determination by the implantable infusion device that the monitored biometric data is indicative of an opioid overdose.

An implantable infusion device in accordance with one embodiment of a present invention includes an outlet port configured to be secured to a catheter, a reservoir configured to store an infusible substance, a fluid transfer device, operably connected to the reservoir and outlet port, configured to transfer the infusible substance from the reservoir to the outlet when actuated, and a controller, operably connected to the fluid transfer device, configured to actuate the fluid transfer device in accordance with a stored delivery profile, to receive monitored biometric data, to determine whether or not the monitored biometric data is indicative of an opioid overdose, and to immediately end actuation of the fluid transfer device in response to a determination by the controller that the monitored biometric data is indicative of an opioid overdose. The present invention also includes systems with one or more of the patient monitoring sensors described herein in combination with such implantable infusion devices.

A method in accordance with one embodiment of a present invention includes the steps of delivering pain medication to a patient with an implantable infusion device, monitoring biometric data of the patient with a patient monitor, determining, with the implantable infusion device, whether or not the monitored biometric data is indicative of pain above a predetermined threshold, increasing delivery of the pain medication with the implantable infusion device in response to a determination by the implantable infusion device that the monitored biometric data is indicative of pain above the predetermined threshold.

An implantable infusion device in accordance with one embodiment of a present invention includes an outlet port configured to be secured to a catheter, a reservoir configured to store an infusible substance, a fluid transfer device, operably connected to the reservoir and outlet port, configured to transfer the infusible substance from the reservoir to the outlet when actuated, and a controller, operably connected to the fluid transfer device, configured to actuate the fluid transfer device in accordance with a stored delivery profile, to receive monitored biometric data, to determine whether or not the monitored biometric data is indicative of pain above a predetermined level, and to increase delivery of the infusible substance in response to a determination by the controller that the monitored biometric data is indicative of pain above the predetermined level.

There are a variety of advantages associated with the present apparatus and methods. By way of example, but not limitation, the present methods and apparatus are able to automatically adjust the dosage being delivered based on the actual state of the patient. The present methods and apparatus are also able to automatically cease delivery of pain medication in response to overdose.

The above described and many other features of the present inventions will become apparent as the inventions become better understood by reference to the following detailed description when considered in conjunction with the accompanying drawings. dr

### BRIEF DESCRIPTION OF THE DRAWINGS

Detailed descriptions of exemplary embodiments will be made with reference to the accompanying drawings.

FIG. 1 is a block diagram of a system in accordance with one embodiment of a present invention.

FIG. 2 is a representation of an implantable infusion device with a catheter that is located within the subarachnoid space.

FIG. 3 is a section view of a catheter that is located with the subarachnoid space.

FIG. 4 is a plan view of an implantable infusion device in accordance with one embodiment of a present invention.

FIG. 5 is a plan view of the implantable infusion device illustrated in FIG. 4 with the cover removed.

FIG. 6 is a partial section view taken along line 6-6 in FIG. 4.

FIG. 7 is a block diagram of the implantable infusion device illustrated in FIGS. 4-6.

FIG. 8 is a graphical illustration of one example of an exemplary delivery profile.

FIG. 9 is a block diagram of a patient monitoring sensor in accordance with one embodiment of a present invention.

FIG. 10 is a side view of a catheter with a patient monitoring sensor in accordance with one embodiment of a present invention.

FIG. 11 is section view taken along line 11-11 in FIG. 10.

FIG. 12 is a side view of a catheter with a patient monitoring sensor in accordance with one embodiment of a present invention.

FIG. 13 is a perspective view of a patient monitoring sensor in accordance with one embodiment of a present invention.

FIG. 14 is a block diagram of the patient monitoring sensor illustrated in FIG. 13.

FIG. 15 is a front view of a patient monitoring sensor in accordance with one embodiment of a present invention.

FIG. 16 is a block diagram of the patient monitoring sensor illustrated in FIG. 15.

FIG. 17 is a plan view of a remote control in a locked state in accordance with one embodiment of a present invention.

FIG. 18 is partial section view taken along line 18-18 in FIG. 17.

FIG. 19 is a plan view of the remote control illustrated in FIG. 17 in an unlocked state.

FIG. 20 is a block diagram showing certain aspects of the remote control illustrated in FIG. 17.

FIG. 21 is a front view of a clinicians programming unit in accordance with one embodiment of a present invention.

FIG. 22 is a block diagram of the clinicians programming unit illustrated in FIG. 21.

FIG. 23 is a front view of a smart phone in accordance with one embodiment of a present invention.

FIG. 24 is a block diagram of the smart phone illustrated in FIG. 23.

FIG. 25 is a flow chart in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to the exemplary implantable infusion device described herein and, instead, are applicable to other implantable infusion devices that currently exist or are yet to be developed.

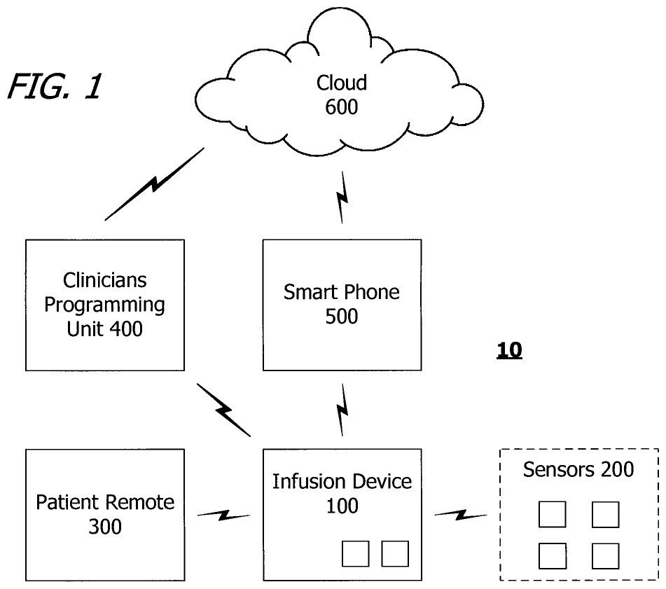

An exemplary system in accordance with one embodiment of a present invention is generally represented by reference numeral 10 in FIG. 1. The exemplary system includes an implantable infusion device (or “infusion device”) 100, one or more patient monitoring sensors 200a-200e that may be used, for example, to provide biometric data that is indicative of an opioid overdose and/or pain (collectively referred to as “patient monitoring sensors 200”), a patient remote control 300, a clinicians programming unit 400, and a smart phone 500, such an IOS or Android smart phone, that may be used to connect the implantable infusion device to the cloud 600. As used herein, an “implantable infusion device” is a device that includes a reservoir and an outlet, and is sized, shaped and otherwise constructed (e.g., sealed) such that both the reservoir and outlet can be simultaneously carried within the patient's body.

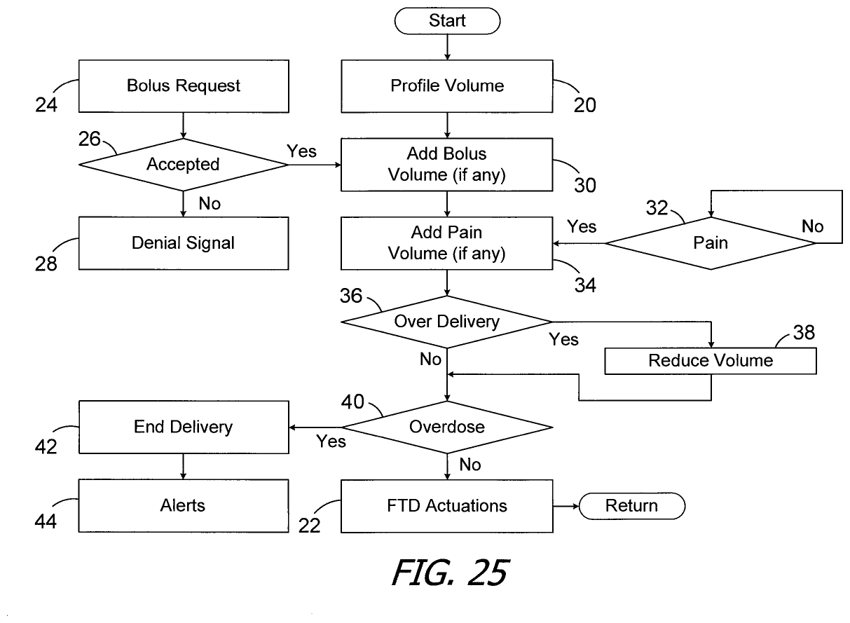

As is discussed in greater detail below with reference to, for example, FIG. 25, the data from the patient monitoring sensors 200 may be used by the infusion device to provide closed-loop control of infusible substance delivery. Such closed-loop control is especially useful in context of pain control, where opioids and non-opioid pain medications are delivered by implantable infusion devices, and the present inventions are discussed primarily in this context. Here, pain medication delivery may be increased by the infusion device 100 based on biometric data from one or more of the patient monitoring sensors 200 that is indicative of pain above a predetermined threshold. Alternatively, or in addition, the infusion device 100 will cease delivery of pain medication delivery in response to biometric data from one or more of the patient monitoring sensors 200 that is indicative of an opioid overdose.

A wide variety of patient monitoring sensors 200 may be employed. In the exemplary context of opioid overdose prevention and/or pain control, such sensors may include sensors that monitor nerve activity and/or the resulting muscle activity near the catheter tip, sensors that monitor chemical signals which are indicative of pain, sensors that monitor respiration (e.g., respiration rate and amplitude), sensors that monitor blood oxygen saturation (402), sensors that monitor blood pressure, sensors that monitor body temperature, and/or sensors that monitor heart rate. Increases in nerve activity and/or the resulting muscle activity near the catheter tip may be indicative of pain. The presence of chemical pain mediators such as prostaglandin, bradykinin, and serotonin released by mast cells may be indicative of pain. Decreases in respiration rate and amplitude may be indicative of an opioid overdose, while increases in respiration rate and amplitude may be indicative of pain. Decreases in blood oxygen saturation may be indicative of opioid overdose. Increases in blood pressure, body temperature, heart rate, and heart rate variability may be indicative of pain, while decreases in blood pressure, body temperature, heart rate, and heart rate variability may be indicative of opioid overdose.

Data from physiological sensors that could be used to assess chronic pain levels include: heart rate variability, accelerometer based activity evaluation, skin conductance, EMG data from muscles known to have been sources of chronic pain, EEG signals and computer vision recognition of facial expressions associated with pain; as discussed in: Naranjo-Hernandez D, Reina-Tosina J, Roa L M., Sensor Technologies to Manage the Physiological Traits of Chronic Pain: A Review, Sensors (Basel). 2020 Jan 8;20(2):365, which is incorporated herein by reference in its entirety. Computer vison facial recognition of pain may be accomplished using a cellphone camera, with images analyzed by an artificial intelligence ((AI) system. Analysis of EEG data to determine pain may also be done by an AI system.

Herat rate could also be used as a surrogate for pain, when accelerometer data is also analyzed to account for increases in heart rate due to pain and not due to exercise.

Data from physiological sensors that could be used to detect opioid use include: electrodermal activity (EDA), skin temperature and tri-axis acceleration data, as discussed in: Mahmud MS, Fang H, Wang H, Carreiro S, Boyer E. Automatic Detection of Opioid Intake Using Wearable Biosensor, International Conference on Computing, Networking and Communications, 2018 March; 2018:784-788, which is incorporated herein by reference in its entirety.

Some patient monitoring sensors may be carried on and/or within the implantable infusion device 100, i.e., on or within the infusion device housing and/or on or within the infusion device catheter. Some patient monitoring sensors may be carried on leads and catheters that do not provide infusible substances and are connected to the implantable infusion device 100. Some patient monitoring sensors, which are configured to communicate wirelessly with the implantable infusion device 100, may be worn on the patient's body. Various examples of patient monitoring sensors are described below with reference to FIGS. 9-16.

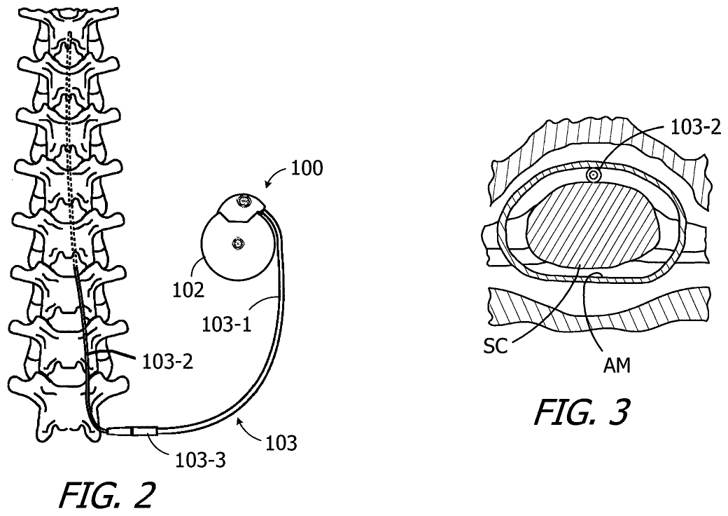

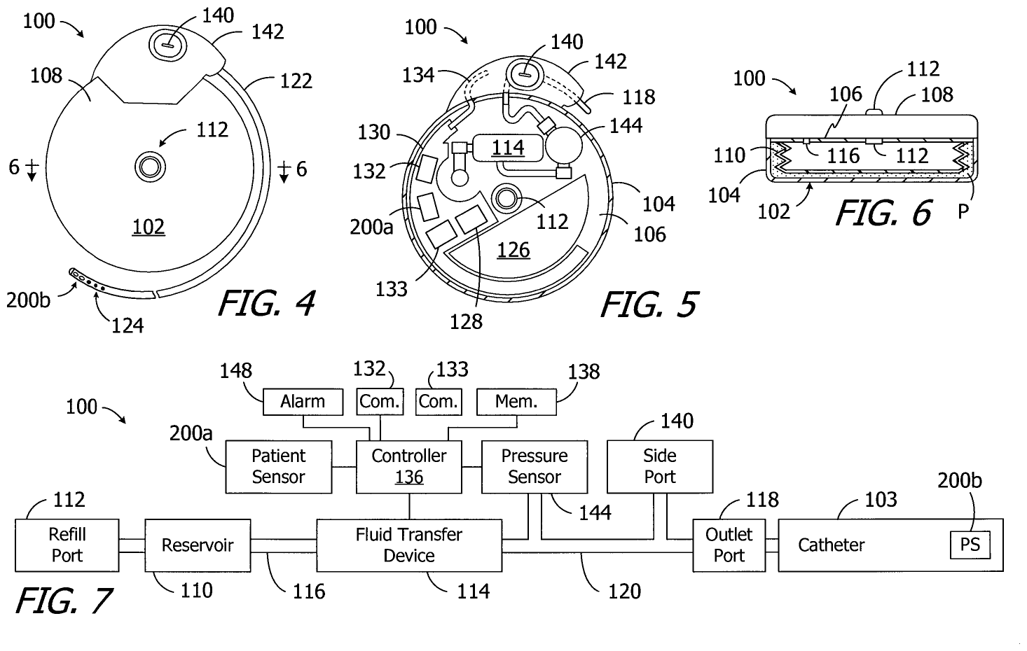

Although the present inventions are not so limited, the exemplary infusion device 100 may be used to deliver infusible substances to the intrathecal space. To that end, and referring to FIGS. 2 and 3, the infusion device 100 includes a housing 102 in which a reservoir, a fluid transfer device and other elements that are contained, as is discussed below with reference to FIGS. 4-7, and a catheter 103. The catheter 103 includes a proximal catheter 103-1, a subarachnoid catheter 103-2, and a connector assembly 103-3. The connector assembly 103-3 may be used to connect the proximal catheter 103-1 to the subarachnoid catheter 103-2 after the subarachnoid catheter has been positioned within the patient's body. For example, in those instances where a stylet is used to push the distal portion of the subarachnoid catheter 103-2 to the target location, the subarachnoid catheter will be connected to the proximal catheter 103-1 after the stylet has been removed. The infusible substance may then be delivered to, for example, the portion of the subarachnoid space between the spinal cord SC and the arachnoid mater AM, as is illustrated in FIG. 3. Exemplary subarachnoid catheters are disclosed in U.S. Pat. Pub. No. 2010/0076407A1, which is incorporated herein by reference in its entirety.

The exemplary infusion device 100 illustrated in FIGS. 3-7 includes a housing 102 (e.g., a titanium housing) with a bottom portion 104, an internal wall 106, and a cover 108. An infusible substance (e.g., a pain medication) may be stored in a reservoir 110 that is located within the housing bottom portion 104. The infusible substance may also be a mixture of two or more infusible substances. The reservoir 110 may be replenished by way of a refill port 112 that extends from the reservoir, through the internal wall 106, to the cover 108. A hypodermic needle (not shown), which is configured to be pushed through the refill port 112, may be used to replenish the reservoir 110.

A wide variety of reservoirs may be employed. In the illustrated embodiment, the reservoir 110 is in the form of a titanium bellows that is positioned within a sealed volume defined by the housing bottom portion 104 and internal wall 106. The remainder of the sealed volume is occupied by propellant P, which may be used to exert negative pressure on the reservoir 110. The negative pressure is advantageous in that it may be used to draw the infusible substance into the reservoir 110 as well as to prevent unintended delivery of the infusible substance when the fluid transfer device fails. Other reservoirs that may be employed in the present infusion devices include reservoirs in which propellant exerts a positive pressure. Still other exemplary reservoirs include negative pressure reservoirs that employ a movable wall that is exposed to ambient pressure and is configured to exert a force that produces an interior pressure which is always negative with respect to the ambient pressure.

The exemplary ambulatory infusion device 100 illustrated in FIGS. 4-7 also includes a fluid transfer device 114. The inlet of a fluid transfer device 114 is coupled to the interior of the reservoir 110 by a passageway 116, while the outlet of the fluid transfer device is coupled to an outlet port 118 by a passageway 120. Operation of the fluid transfer device 114 causes infusible substance to move from the reservoir 110 to the outlet port 118. The catheter 103 may be connected to the outlet port 118 so that the infusible substance passing through the outlet port will be delivered to the subarachnoid space or other target body region in spaced relation to the infusion device 100 by way of outlets 124 near the end of the catheter.

A wide variety of fluid transfer devices may be employed. In the illustrated embodiment, the fluid transfer device 114 is in the form of an electromagnetic pump. The present inventions are not, however, limited to electromagnetic pumps and may include other types of fluid transfer devices. Such devices include, but are not limited to, other electromagnetic pumps, solenoid pumps, piezo pumps, and any other mechanical or electromechanical pulsatile pump. In the exemplary context of implantable drug delivery devices, the electromagnetic pump will typically deliver about 0.25 microliter per actuation of the electromagnet, and each actuation may take about 3 milliseconds to complete. Additionally, although the exemplary fluid transfer device 114 is provided with internal valves (e.g., a main check valve and a bypass valve), valves may also be provided as separate structural elements that are positioned upstream of and/or downstream from the associated fluid transfer device.

It should be noted that, in other implementations, the reservoir may include two or more compartments that may be used to store different infusible substances for delivery to the same or different body sites. Alternatively, or in addition, at least two fluid transfer devices for respectively transferring first and second infusible substance flow to the same or different body sites may be provided. Additional details concerning the use of multiple reservoir compartments and multiple fluid transfer devices are provided in U.S. Pat. No. 8,002,747, which is incorporated herein by reference in its entirety.

Energy for the fluid transfer device 114, as well for other aspects of the exemplary infusion device 100, is provided by the battery 126 illustrated in FIG. 5. In the specific case of the fluid transfer device 114, the battery 126 is used to charge one or more capacitors 128 and is not directly connected to the fluid transfer device itself. The capacitor(s) 128 are connected to an electromagnetic coil in the fluid transfer device 114, and disconnected from the battery 126, when the electromagnetic coil is being energized, and are disconnected from the electromagnetic coil and connected to the battery when the capacitor(s) are being recharged and/or when the fluid transfer device is at rest. The capacitor(s) 128 are carried on a board 130. A wireless communication device 132, which is connected to an antenna 134, is carried on the same side of the board 130 as the capacitor(s) 128. The exemplary communication device 132 is a Bluetooth communication device. An RF communication device 133 and associated antenna (not shown) may also be provided. Communication devices 132 and 133 may also share a common antenna. Other suitable communication devices include, but are not limited to, oscillating magnetic field communication devices, static magnetic field communication devices, optical communication devices, ultrasound communication devices and direct electrical communication devices.

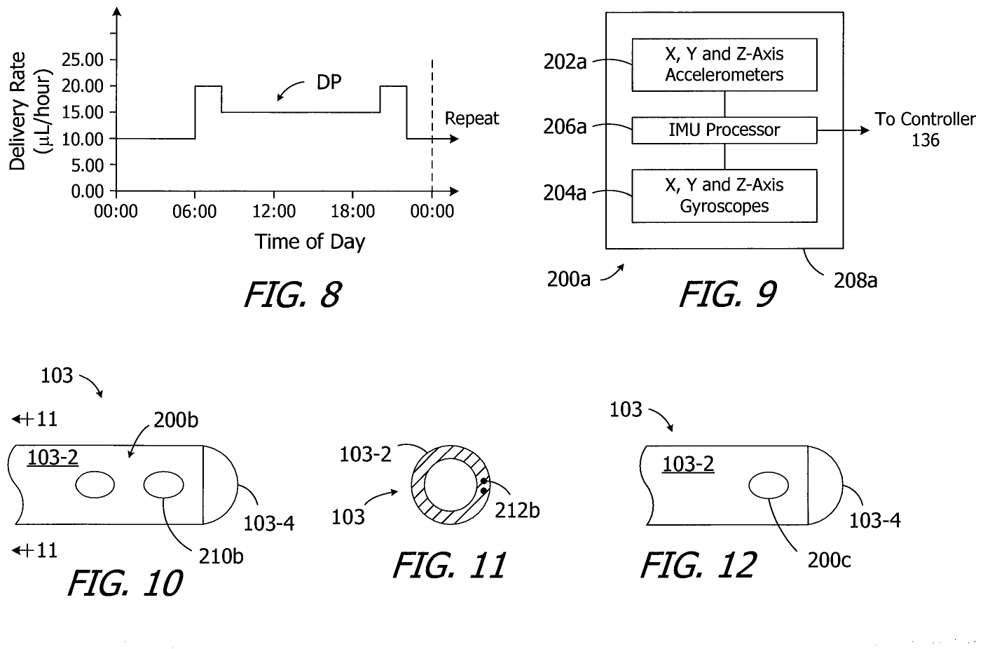

A controller 136 (FIG. 7), such as a microprocessor, microcontroller (e.g., the Texas Instruments MSP 430 microcontroller) or other control circuitry, is carried on the other side of the board 130. The controller controls the operations of the infusion device 100 in accordance with instructions stored in memory 138 and/or provided by an external device by way of the communication device 132 or 133. For example, the controller 136 may be used to control the fluid transfer device 114 to supply an infusible substance to the patient in accordance with, for example, a stored basal delivery profile. The exemplary delivery profile DP illustrated in FIG. 8, which has a twenty-four hour cycle time and may be expressed in terms of the volume delivered per hour. Other exemplary delivery profiles may include the same delivery rate over the 24-hour period and/or time periods with no delivery. The controller 136 may be used to control the fluid transfer device 114 to supply additional infusible substance in response to a bolus delivery request from the patient remote control 300, or to deny the bolus delivery request in predetermined instances (e.g., where the bolus deliver would result in over-delivery). The controller 136 may also be used to increase, decrease or suspend delivery of an infusible substance in response to one or more sensed physiologic surrogates for pain, as is described in greater detail below with reference to FIG. 25.

Referring to FIGS. 4, 5 and 7, the exemplary infusion device 100 is also provided with a side port 140 that is connected to the passageway 120 between the outlet of the fluid transfer device 114 and the outlet port 118. The side port 140 facilitates access to an implanted catheter 103, typically by way of a hypodermic needle. For example, the side port 140 allows clinicians to push fluid into the catheter 103 and/or draw fluid from the catheter for purposes such as checking catheter patency, sampling CSF, injecting contrast dye into the patient and/or catheter, removing medication from the catheter prior to dye injection, injecting additional medication into the region at the catheter outlet 124, and/or removing pharmaceuticals or other fluids that are causing an allergic or otherwise undesirable biologic reaction.

The outlet port 118, a portion of the passageway 120, the antenna 134 and the side port 140 are carried by a header assembly 142. The header assembly 142 is a molded, plastic structure that is secured to the housing 102. The housing 102 includes a small aperture through which portions of the passageway 120 are connected to one another, and a small aperture through which the antenna 134 is connected to the board 130.

The exemplary infusion device 100 illustrated in FIGS. 4-7 also includes a pressure sensor 144 that is connected to the passageway 120 between the outlet of the fluid transfer device 114 and the outlet port 118. As such, the pressure sensor 144 senses the pressure at the outlet port 118 which, in the illustrated embodiment, is also the pressure within the catheter 103. The pressure sensor 144 is connected to the controller 136 and may be used to analyze a variety of aspects of the operation of the exemplary implantable infusion device 100. For example, pressure measurements may be used to determine whether or not the fluid transfer device 114 is functioning properly and whether or not there is a complete or partial blockage in the catheter 103. Pressure measurements may also be used to determine whether or not the side port 140 has been accessed. The controller 136 may perform a variety of different functions in response to determination that the fluid transfer device 114 is not functioning properly, the catheter 103 is blocked, and/or the side port 140 has been accessed. For example, the controller 136 may actuate an audible alarm 148 that is located within the housing 102 in order to signal that the fluid transfer device 114 is not functioning properly, the catheter 103 is blocked, and/or that the side port 140 has been accessed.

As noted above, a wide variety of patient monitoring sensors may be employed. Referring first to FIG. 9, the exemplary patient monitoring sensor 200a is an inertial monitoring unit (“IMU”) that may be used to monitor respiration rate and amplitude by monitoring movement in the chest area. The exemplary IMU 200a, which may be located within the infusion device housing 102, includes X, Y and Z-axis accelerometers 202a and X, Y and Z-axis gyroscopes 204a that are connected to a processor 206a located within a housing 208a. The IMU processor 206a provides measured movement data to the infusion device controller 136, which correlates the movement data to respiration rate and amplitude. The infusion device controller 136, in turn, determines whether or not the sensed respiration rate and amplitude show respiratory depression indicative of an opioid overdose, and proceeds accordingly, in the manner described below with reference to FIG. 25. The infusion device controller 136 may also use data from the IMU processor 206a to produce a seismocardiogram (“SCG”). Heart rate and heart rate variability, which are also indicative of pain as well as opioid overdose, can be extracted from the SCG by the infusion device controller 136.

Another exemplary patient monitoring sensor is generally represented by reference numeral 200b in FIG. 10. Here, the sensor 200b consists of a pair of electrical contacts 210b. The electrical contacts 210b may be carried on the exterior of the catheter 103. In the illustrated embodiment, the electrical contacts 210b are carried on the distal portion of the subarachnoid catheter 103-2 near the tip 103-4. In other implementations, one or both of the electrical contacts 210b may be located on the tip 103-4. Power and return wires 212b extend from the electrical contacts 210b to the infusion device housing 102 and are operably connected to the infusion device controller 136. One contact 212b may function as the sensing contact, while the other functions as the return. The sensor 200b is configured to sense nerve activity and/or the resulting muscle activity near the catheter tip 103-4. The infusion device controller 136 determines whether or not the sensed nerve and/or resulting muscle activity is indicative of pain, and proceeds accordingly, in the manner described below with reference to FIG. 25.

Turning to FIG. 12, the patient monitoring sensor 200c is a chemical sensor which senses chemical signals indicative of pain, such as the presence of pain mediators such as prostaglandin, bradykinin, and serotonin. In the illustrated embodiment, the patient monitoring sensor 200c is carried near the distal end of the subarachnoid catheter 103-2 near the tip 103-4. In other implementations, sensor 200c may be located on the tip 103-4. One or more wires (not shown) extend from the sensor 200c to the infusion device housing 102 and are operably connected to the infusion device controller 136. The infusion device controller 136 determines whether or not the sensed chemical signals are indicative of pain, and proceeds accordingly, in the manner described below with reference to FIG. 25.

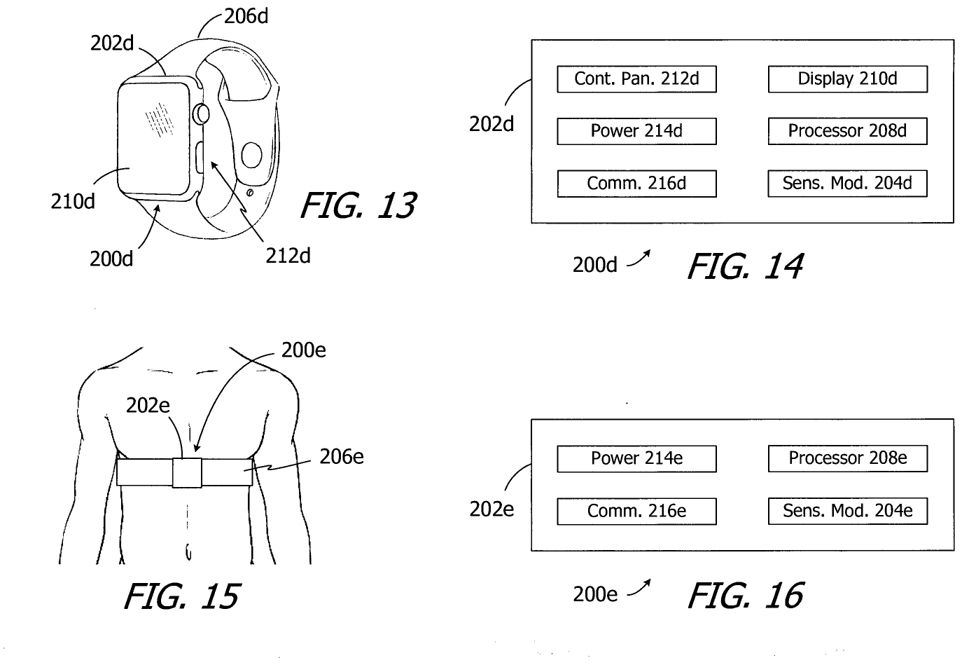

Wearable patient monitoring sensors, i.e. external sensors that are positioned against skin, may also be employed. One example of a wearable patient monitoring sensor is generally represented by reference numeral 200d in FIGS. 13 and 14. The exemplary patient monitoring sensor 200d, which is configured to be worn on the patient's arm at the wrist, includes a housing 202d, a sensor module 204d that is associated with the housing, and a wrist band 206d that secures the housing (and sensor module) against the patient's skin. Other elements include a processor 208d, a display 210d, a control panel 212d, a rechargeable battery or other power supply 214d, and a communication device 216d (e.g., a Bluetooth communication device). The sensor module 204d includes one or more sensors configured to sense biometric data corresponding to blood oxygen saturation (SpO2), blood pressure, body temperature, heart rate and/or heart rate variability. The infusion device controller 136 receives sensed biometric data by way of the communication devices 132 and 216d, and may then determine whether or not the sensed biometric data are indicative of pain or opioid overdose, and proceeds accordingly, in the manner described below with reference to FIG. 25. Additional details concerning wrist worn patient monitoring sensors may be found in, for example, U.S. Pat. Pub. No. 2020/0260972A1, which is incorporated herein by reference in its entirety.

Another exemplary wearable patient monitoring sensor is generally represented by reference numeral 200e in FIGS. 15 and 16. The exemplary patient monitoring sensor 200e, which is configured to be worn on the patient's chest, includes a housing 202e, a sensor module 204e that is associated with the housing, and a chest band 206d that secures the housing (and sensor module) against the patient's skin. Other elements include a processor 208d, a rechargeable battery or other power supply 214d, and a communication device 216d (e.g., a Bluetooth communication device). The sensor module 204d includes one or more sensors configured to sense biometric data corresponding to respiration rate, blood oxygen saturation (SpO2), blood pressure, body temperature, heart rate and/or heart rate variability. The infusion device controller 136 receives sensed biometric data by way of the communication devices 132 and 216e, and may then determine whether or not the sensed biometric data are indicative of pain or opioid overdose, and proceeds accordingly, in the manner described below with reference to FIG. 25. The patient monitoring sensor 200e does not, in the illustrated implementation, include a display or a control panel. A clinicians programming unit (e.g., clinicians programming unit 400) or a communication device (e.g., communication device 500) may be used to control the patient monitoring sensor 200e. Additional details concerning chest worn patient monitoring sensors may be found in, for example, U.S. Pat. Pub. No. 2020/0312453A1, which is incorporated herein by reference in its entirety.

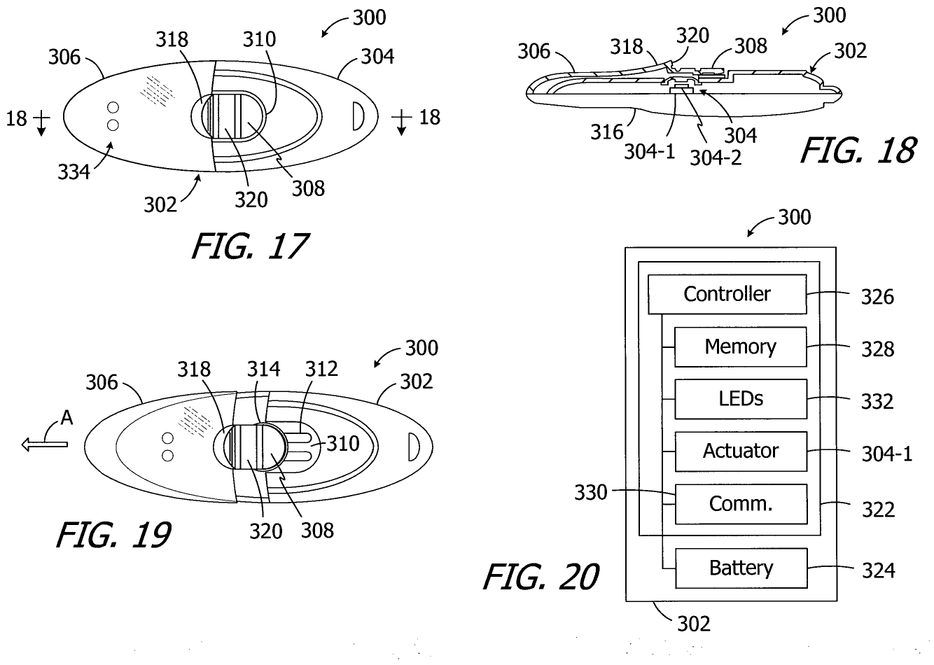

One example of a patient remote control 300 is illustrated in FIGS. 17-20. The exemplary remote control 300 includes a housing 302 and a button 304 that has an actuator 304-1 with a movable element 304-2. The actuator 304-1 may be, for example, a normally open switch that is biased to the open state and is closed in response to downward (in the illustrated orientation) movement of the movable element 304-2. The housing 302 carries a movable button control element 306 with a depressible member 308 that is positioned over the button 304. The remote control 300 will generate a bolus delivery signal when the button 304 is pressed and, depending on its position, the button control element 306 will either prevent or allow the button to be pressed. To that end, the remote control 300 is shown in the locked state, i.e. the state in which the button 304 may not be pressed, in FIGS. 17 and 18. The depressible member 308 is aligned with a barrier 310, which may include abutments 312, that prevents the depressible member 308 on the button control element 306 from being depressed, thereby preventing the button 304 from being pressed. The remote control 300 may be adjusted to the unlocked state, where the button 304 may be pressed, by moving the button control element 306 in the direction of arrow A until the depressible member 308 is no longer aligned with the barrier 310 and is instead aligned with a housing aperture 314 (FIG. 19). The housing 302 may include a surface 316 that is shaped to receive the user's forefinger and the button control element 306 may include a raised area 318 that combines with the depressible member 308 to form a region that is shaped to receive the user's thumb. Ridges 320 prevent the user's thumb from slipping. Once the button control element 306 has reached the unlocked position illustrated in FIG. 19, the user will be able to press the button 304 by depressing the depressible member 308.

Referring more specifically to FIG. 20, the patient remote control 300 also includes a circuit board 322 and a battery 324. The circuit board 322 carries a controller 326, memory 328, and a communication device 330 (including an antenna), such as a Bluetooth or RF communication device, that is configured to communicate with the pump communication device 132 or 133. The circuit board 322 also carries the actuator 304-1 and a pair of LEDs 332 (or other light emitting elements) that are visible through openings 334. The LEDs 332, which may be the same color or different colors (e.g., green and red), may be used to communicate various diagnostic issues (e.g., a low battery) as well as the other issues such as a bolus request denial. Additional information concerning exemplary patient remote controls is provided in U.S. Pat. Nos. 8,352,041 and 9,135,810, which are incorporated herein by reference in their entireties.

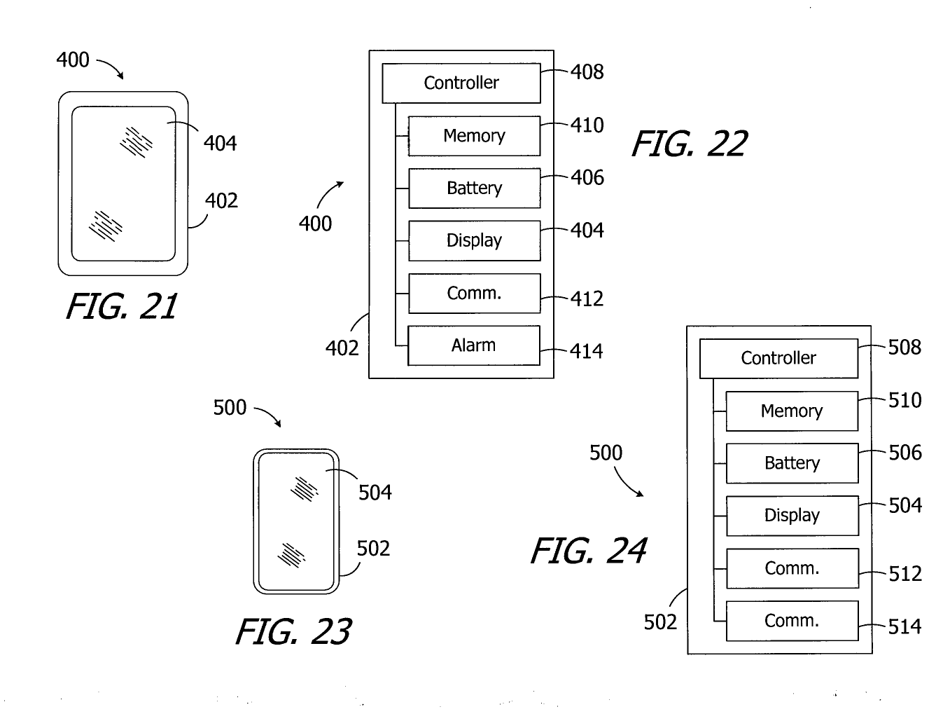

Turning to FIGS. 21 and 22, the exemplary clinicians programming unit 400 includes a housing 402, a touch screen display 404 (or other input device, such as a keypad, with or without a separate display), a battery or other power source 406, a controller 408, such as a microprocessor, microcontroller or other control circuitry, memory 410, and a communication device 412 (including an antenna), such as a Bluetooth or RF communication device (or both), that is configured to send signals to and receive signals from the communication device 132 (or 133) in the implantable infusion device 100 and to connect the clinicians programming unit to communication networks. In some instances, the remote control may also include an audible alarm 414. The exemplary clinicians programming unit 400 may be used to perform a variety of conventional control functions including, but not limited to, turning the infusion device ON or OFF and programming various infusion device parameters. Examples of such parameters include, but are not limited to, the rate of medication delivery during a particular time period, the time at which delivery of a medication is to commence or change, and the time at which delivery of a medication is to end. Additionally, in at least some implementations, the implantable infusion device 100 will transmit signals to the clinicians programming unit 400. The signals may include data from the patient monitoring sensors 200 and/or the interpretation thereof by the controller 136 (including pain and overdose determinations). The signals may also provide status information about the infusion device 100 that may be stored in memory 410 and/or displayed on the display 404. Examples of such status information include, but are not limited to, the state of charge of the battery 126, the amount of medication remaining in the reservoir 110, the amount of medication that has been delivered during a specified time period, and the presence of a catheter blockage. The clinicians programming unit 400 may also be connected to an appropriate communication network and used to transmit stored data to, for example, cloud computing facilities for analysis.

Referring to FIGS. 23 and 24, and although the present inventions are not so limited, the exemplary smart phone 500 includes a housing 502, a touch screen display 504, a battery or other power source 506, a controller 508, such as a microprocessor, microcontroller or other control circuitry, memory 510, and a communication device 512 (including one or more antennas), such as Bluetooth and/or RF communication devices (or both), that is configured to send signals to and receive signals from the communication device 132 (or 133) in the implantable infusion device 100, a cellular communication device 512 (including an antenna), as well as other conventional features such as a microphone and speaker (not shown). The smart phone may be used to transmit signals, such as bolus request or request for information, to the implantable infusion device 100. The implantable infusion device 100 will also transmit signals to the exemplary smart phone 500 that may be stored in memory 510 and/or displayed on the display 504. The signals may include data from the patient monitoring sensors 200. The signals may also provide status information about the infusion device 100. Examples of such status information include, but are not limited to, the state of charge of the battery 126, the amount of medication remaining in the reservoir 110, the amount of medication that has been delivered during a specified time period, and the presence of a catheter blockage. The smart phone 500 may also be connected to an appropriate communication network and used to transmit stored data to, for example, cloud computing facilities for analysis. Additionally, smart phone 500 may also be used to send a message, such as text message, to one or more predesignated persons (e.g., the patient, a caregiver, a family member, a medical professional and/or emergency personnel) alerting them to overdose.

Turning to FIG. 25, the controller 136 may control the exemplary implantable infusion pump 100 to operate as follows in the exemplary context of pain medication. The volume of pain medication to be delivered to the patient at any particular time in the delivery cycle is initially defined by the delivery profile such as the profile illustrated in FIG. 8 (Step 20). In at least some instances, delivery will occur at the beginning of each minute of the delivery cycle and, absent any adjustments, the number of fluid transfer device actuations (Step 22) at the beginning of each minute will correspond to the delivery profile volume associated with that minute. The volume delivered may also be adjusted by the controller 136 as necessary.

One delivery adjustment that may occur is the delivery of additional pain medication in response to a bolus request from the patient remote control 300 or smart phone 500 (Step 24). The bolus request will either be denied or accepted by the controller 136 (Step 26). The bolus request may be denied, for example, if the associated delivery profile (or portion thereof) does not permit bolus requests or if the permitted number of boluses in a given time period has been surpassed. A denial signal will be sent to the patient remote control 300 or smart phone 500 by the infusion pump 100 (Step 28), and the remote control or smart phone will provide an indication of the denial. An accepted bolus request will result in the bolus volume being added to the next delivery associated with the delivery profile (Step 30). The additional volume may be produced with additional actuations of the fluid transfer device 114 in Step 22.

Another delivery adjustment that may occur is the delivery of additional pain medication in response to a determination by the controller 136, based biometric data from one or more of the patient monitoring sensors 200, that the patient is experiencing pain above the predetermined threshold (Step 32). The volume of additional pain medication provided in those instances where the experienced pain exceeds the threshold may be the same in all instances or may vary based on the difference between the threshold and the experienced pain. In either case, the volume will be added to the next delivery associated with the delivery profile (Step 34). The additional volume may be produced with additional actuations of the fluid transfer device 114 in Step 22.

It should also be noted that, in some instances, the additional volume associated with the bolus request and/or sensed pain could result in over delivery of pain medication to the patient. Accordingly, the controller 136 will compare the total adjusted volume to be delivered to a predetermined maximum volume (Step 36) and, if necessary reduce the volume to be delivered to the predetermined maximum volume. The reduction in volume may be produced by reducing the number of fluid transfer device actuations in Step 22.

Still another adjustment is the cessation of pain medication delivery in response to a determination (Step 40) by the controller 136, based biometric data from one or more of the patient monitoring sensors 200, that the patient is suffering from an opioid overdose. Here, regardless of the pain medication volume dictated by the delivery profile, bolus request, and/or experienced pain, the controller 136 will immediately end delivery of the pain medication (Step 42). The controller 136 will also instruct the smart phone 500 to transmit a message (Step 44), such as a text message, to one or more predesignated persons or organization (e.g., the patient, a caregiver, a family member, a medical professional and/or emergency responders) alerting them to the overdose. In at least some implementations, delivery of pain medication will not restart without permission from a clinician by way of the clinicians programming unit 400 or from a remote location by way of the smart phone 500.

Baseline values of the biometric data described above against which the sensed values are compared may be established in a variety of ways. For example, because pain is a subjective measure that varies across patient populations, values associated with pain detection may be derived preclinical and clinical studies using patient pain inputs 1 to 10 visual analog scale that are correlated to the amount of nerve and muscle activity, the levels of chemical pain mediators such as prostaglandin, bradykinin and serotonin, respiration rate, blood pressure, body temperature and heart rate and heart rate variability in preclinical and clinical studies. The correlations set in preclinical and clinical studies may also be adjusted based on patient experience. Similarly, with respect to overdose, baseline values for respiration rate, blood oxygen saturation, blood pressure, body temperature and heart rate and heart rate variability may be established in preclinical and clinical studies and correlated to the patient's age, sex, weight, heath conditions and the like.

Machine learning algorithms, such as deep neural networks, as well as other classical machine learning algorithms, such as logistic regression, can be employed to classify patterns of data from the sensors 200. These algorithms will provide probability scores on the sensed levels of pain and respiratory distress. Data from the pre-clinical and clinical studies of patient implanted with the implantable pump can be uploaded to the cloud from a smartphone. Cloud based computing can be used to further refine and test machine learning models across a wide range of patient populations. As more data becomes available, more sophisticated machine learning models using ensemble/boosting techniques can be used to further refine the algorithms for delivery of drugs to individual patients. Ensemble learning involves implementing multiple classification algorithms, each voting on a decision. Majority voting is used to arrive at a final decision. Such methods can provide a higher degree of accuracy that is required for a closed-loop, demand-based, drug delivery system.

Deep learning may also be used in closing the loop with respect to automatically adjusting drug delivery. The neural network will learn the titration of the drug for each patient. It can also learn to predict at what times the drug(s) need to be delivered for each patient, and to adjust the delivery profile as appropriate, so that the system does not wait for the patient to be in pain before delivering therapy. As part of the learning process, a reduction in pain level as a feedback mechanism will be used to decide if the adjustments to drug delivery are improving the overall pain score. In addition to directly measuring surrogate data for pain, the system can adjust the drug titration with some activity the patient participates in during the day (e.g., vigorous exercise) that causes pain. Respiration and activity data from inertial sensors built into the pump could help identify these patterns of activity so that drug delivery can be adjusted.

The data and learning being deposited into a database from the clinicians programmer 400 and then aggregated with the data from other infusion device patients to facilitate better understanding of trends, behavior, etc. by public health professionals and doctors, thereby facilitating the development of improved treatment regimens based on the type of drug being delivered for that particular disease.

Although the inventions disclosed herein have been described in terms of the preferred embodiments above, numerous modifications and/or additions to the above-described preferred embodiments would be readily apparent to one skilled in the art. By way of example, but not limitation, the present inventions have application in other types of ambulatory infusion device, such as patch pumps and other externally carried infusion devices. Wireless sensors that are implanted into the body may be employed. It is intended that the scope of the present inventions extend to all such modifications and/or additions and that the scope of the present inventions is limited solely by the claims set forth below.

We claim:

1. A method, comprising the steps of:
delivering pain medication to a patient with an implantable infusion device;
monitoring biometric data of the patient with a patient monitor;
determining, with the implantable infusion device, whether or not the monitored biometric data is indicative of an opioid overdose; and
immediately ending delivery of the pain medication with the implantable infusion device in response to a determination by the implantable infusion device that the monitored biometric data is indicative of an opioid overdose.

2. A method as claimed in claim 1, further comprising the step of:
transmitting an opioid overdose alert in response to a determination by the implantable infusion device that the monitored biometric data is indicative of an opioid overdose.

3. A method as claimed in claim 1, wherein
the monitored biometric data comprises one or more of respiration rate, blood oxygen saturation (SpO2), blood pressure, body temperature, heart rate, heart rate variability, accelerometer data, skin conductance, EMG, EEG and facial expressions.

4. A method as claimed in claim 1, wherein
the implantable infusion device includes housing, a reservoir within the housing, and a fluid transfer device within the housing; and
monitoring biometric data comprises monitoring biometric with a sensor that is within the housing.

5. A method as claimed in claim 1, wherein
monitoring biometric data comprises monitoring biometric data with a body worn sensor; and
the method further comprises wirelessly transmitting the monitored biometric data from the body worn sensor to the implantable infusion device.

6. A method as claimed in claim 1, further comprising the steps of
determining, with the implantable infusion device, whether or not the monitored biometric data is indicative of pain above a predetermined threshold; and
increasing delivery of the pain medication with the implantable infusion device in response to a determination by the implantable infusion device that the monitored biometric data is indicative of pain above the predetermined threshold.

7. An implantable infusion device, comprising:
an outlet port configured to be secured to a catheter;
a reservoir configured to store an infusible substance;
a fluid transfer device, operably connected to the reservoir and outlet port, configured to transfer the infusible substance from the reservoir to the outlet when actuated; and
a controller, operably connected to the fluid transfer device, configured to actuate the fluid transfer device in accordance with a stored delivery profile, to receive monitored biometric data, to determine whether or not the monitored biometric data is indicative of an opioid overdose, and to immediately end actuation of the fluid transfer device in response to a determination by the controller that the monitored biometric data is indicative of an opioid overdose.

8. An implantable infusion device as claimed in claim 7, further comprising:
a wireless communication device operably connected to the controller;
wherein the controller is configured to transmit an opioid overdose alert by way of the wireless communication device in response to a determination that the monitored biometric data is indicative of an opioid overdose.

9. An implantable infusion device as claimed in claim 7, wherein
the monitored biometric data comprises one or more of respiration rate, blood oxygen saturation (SpO2), blood pressure, body temperature, heart rate, heart rate variability, accelerometer data, skin conductance, EMG, EEG and facial expressions.

10. An implantable infusion device as claimed in claim 7, further comprising;
a housing in which the reservoir and fluid transfer device are located; and
a sensor configured to monitor biometric data located within the housing and operably connected to the controller.

11. An implantable infusion device as claimed in claim 7, further comprising:
a wireless communication device operably connected to the controller;
wherein the controller is configured to receive the biometric data by way of the wireless communication device.

12. An implantable infusion device as claimed in claim 7, wherein
the controller is configured to determine whether or not the monitored biometric data is indicative of pain above a predetermined level and to increase actuation of the fluid transfer device in response to a determination by the controller that the monitored biometric data is indicative of pain above the predetermined level.

13. A method, comprising the steps of:
delivering pain medication to a patient with an implantable infusion device;
monitoring biometric data of the patient with a patient monitor;
determining, with the implantable infusion device, whether or not the monitored biometric data is indicative of pain above a predetermined threshold; and
increasing delivery of the pain medication with the implantable infusion device in response to a determination by the implantable infusion device that the monitored biometric data is indicative of pain above the predetermined threshold.

14. A method as claimed in claim 13, wherein
the monitored biometric data comprises one or more of nerve activity, muscle activity, chemical signals, respiration rate, blood pressure, body temperature, heart rate, and heart rate variability.

15. A method as claimed in claim 13, wherein
the implantable infusion device includes housing, a reservoir within the housing, and a fluid transfer device within the housing; and
monitoring biometric data comprises monitoring biometric with a sensor that is within the housing.

16. A method as claimed in claim 13, wherein
monitoring biometric data comprises monitoring biometric data with a body worn sensor; and
the method further comprises wirelessly transmitting the monitored biometric data from the body worn sensor to the implantable infusion device.

17. A method as claimed in claim 13, wherein
monitoring biometric data comprises monitoring biometric data with a sensor on a catheter.

18. A method as claimed in claim 13, further comprising the steps of
determining, with the implantable infusion device, whether or not the monitored biometric data is indicative of an opioid overdose; and
immediately ending delivery of the pain medication with the implantable infusion device in response to a determination by the implantable infusion device that the monitored biometric data is indicative of an opioid overdose.

19. An implantable infusion device, comprising:
an outlet port configured to be secured to a catheter;
a reservoir configured to store an infusible substance;
a fluid transfer device, operably connected to the reservoir and outlet port, configured to transfer the infusible substance from the reservoir to the outlet when actuated; and
a controller, operably connected to the fluid transfer device, configured to actuate the fluid transfer device in accordance with a stored delivery profile, to receive monitored biometric data, to determine whether or not the monitored biometric data is indicative of pain above a predetermined level, and to increase delivery of the infusible substance in response to a determination by the controller that the monitored biometric data is indicative of pain above the predetermined level.

20. An implantable infusion device as claimed in claim 19, wherein
the monitored biometric data comprises one or more of nerve activity, muscle activity, chemical signals, respiration rate, blood pressure, body temperature, heart rate, and heart rate variability.

21. An implantable infusion device as claimed in claim 19, further comprising;
a catheter connected to the outlet port; and
a sensor configured to monitor biometric data carried by the catheter and operably connected to the controller.

22. An implantable infusion device as claimed in claim 19, further comprising;
a housing in which the reservoir and fluid transfer device are located; and
a sensor configured to monitor biometric data located within the fluid transfer device housing and operably connected to the controller.

23. An implantable infusion device as claimed in claim 19, further comprising:
a wireless communication device;
wherein the controller is configured to receive the biometric data by way of the wireless communication device.

24. An implantable infusion device as claimed in claim 19, wherein
the controller is configured to determine whether or not the monitored biometric data is indicative of an opioid overdose, and to immediately end actuation of the fluid transfer device in response to a determination by the controller that the monitored biometric data is indicative of an opioid overdose.
